(19) United States
(12) Patent Application Publication
(10) Pub. No.: US 2014/0356396 A1
(43) Pub. Date: Dec. 4, 2014

(54) ROTAVIRUS PREPARATIONS WITH EXCESS CALCIUM IONS AND HIGH VISCOSITIES THAT ENSURE VIABILITY AT ELEVATED TEMPERATURES

(71) Applicant: Brian L. Pulliam, Cambridge, MA (US)

(72) Inventor: Brian L. Pulliam, Cambridge, MA (US)

(21) Appl. No.: 14/347,237

(22) PCT Filed: Aug. 25, 2012
(86) PCT No.: PCT/US12/52407

§ 371 Date: Aug. 19, 2014

Related U.S. Application Data
(60) Provisional application No. 61/527,439, filed on Aug. 25, 2011.

(51) Int. Cl. A61K 39/15; C12N 7/00; A61K 47/02

(52) U.S. Cl. CPC A61K 39/15; A61K 47/02; C12N 7/00; A61K 2039/525
USPC 424/215.1

(57) ABSTRACT

The invention describes a set of formulations and methods that provide for stabilization of viruses in liquid and dried states. In particular, formulations include Rotavirus preparations with excess Ca2+ and high viscosities that ensure infective potency at elevated temperatures. Methods include bulk purification of Rotavirus from cell culture and administration of formulations as vaccines including components for gastric neutralization.

### FIELD OF INVENTION

The invention relates to methods for stabilization of viruses in liquid or dried states. More particularly, the present invention relates to methods for formulating rotavirus preparations with excess Ca2+ and high viscosities that ensure vaccine viability at elevated temperatures.

### BACKGROUND OF THE INVENTION

A vaccine is a biological preparation that improves immunity to a particular disease. A vaccine typically contains an agent that resembles a disease-causing microorganism, and is often made from attenuated forms of the microbe or its toxins. The agent stimulates the body's immune system to recognize the agent as foreign and to “remember” it so that the immune system can more easily recognize and destroy any of these microorganisms that it later encounters.

Resources required to keep vaccines within a restricted temperature range from the manufacturing point to the point of administration are so great that delivering vaccines to remote areas to treat outbreaks of disease has remained cost prohibitive.

Rotavirus A, which accounts for more than 90% of rotavirus gastroenteritis in humans, is endemic worldwide. At present, available rotavirus vaccines are oral vaccines requiring refrigeration. A need exists for a rotavirus vaccine preparation which is more stable and which removes the need for a “cold chain.” This would allow for more effective large scale distribution of the rotavirus vaccine in remote areas where populations are more likely to be severely affected by a rotavirus outbreak and where access to refrigeration is scarce.

Each year rotavirus causes millions of cases of diarrhea in developing countries, resulting in almost 2 million hospitalizations and an estimated 611,000 deaths. In the United States alone, before initiation of the rotavirus vaccination program, which resulted in over 2.7 million cases of rotavirus gastroenteritis, 60,000 children hospitalized and around 37 deaths occurred annually.

In 1998, a rotavirus vaccine was licensed for use in the United States. Clinical trials in the United States, Finland, and Venezuela had found it to be 80 to 100% effective at preventing severe diarrhea caused by rotavirus A, and researchers had detected no statistically significant serious adverse effects. The manufacturer, however, withdrew it from the market in 1999, after it was discovered that the vaccine may have contributed to an increased risk for intussusception, a type of bowel obstruction, in one of every 12,000 vaccinated infants. The experience provoked intense debate about the relative risks and benefits of a rotavirus vaccine. In 2006, two new vaccines against rotavirus A infection were shown to be safe and effective in children, and in June 2009 the World Health Organization recommended that rotavirus vaccination be included in all national immunization programs to provide protection against this virus.

The rotavirus replicates in the cytoplasm of the host cell. Virions enter the host cell by endocytosis and viral mRNA is transcribed using the viral RNA polymerase that is already present in the virion to form structural protein units of the capsid. The mRNA segments are assembled into the immature capsid and then replicated to form the double stranded RNA genome.

During the replication, rotavirus ligands on the outer capsid hemagglitinating protein VP4 bind to sialic acid receptors on the cell about to be infected. A cleavage of VP4 by trypsin is required for the virion to enter the cytoplasm, the site of rotavirus replication. This occurs when the virion makes direct contact with the cytoplasm. The rough endoplasmic reticulum retains the outer capsid lycoprotein VP7. With the help of a nonstructural glycoprotein, the rotavirus precursor buds off into the cisternae of the rough endoplasmic reticulum and acquires a temporary envelope which is later removed so that the entire outer capsid can be assembled by VP7. The inclusion bodies are formed 6-7 hours after infection in infected cells.

The use of Ca2+ in formulations to stabilize rotavirus is known. However, as will be discussed below, the prior art's discloses using of Ca2+ as a filler, a divalent cation which might be substituted by other divalent cations or as a ratio with Zn2+. Some are completely silent on Ca2+. A few have attempted to use Ca2+ in preparation solution or in the form of CaCo₃ as an anti-neutralization agent. Ultimately, each of these teaches away from the effectiveness of Ca2+ in the stability of the outer capsid of the rotavirus at high temperature (above room temperature). Further, none of the prior art discloses applying excess Ca2+ in formulation to effectively increase the structural stability of the rotavirus.

PCT Application No PCT/GB/84/00268 to Davis et al. discloses methods for preparing an orally-administered immunogenic composition containing live protozoa, such as coccidia, which is transmitted in a cyst stage within an alginate beadlet. The parasite emerges from the cyst after ingestion by a host. The live attenuated parasite in an encysted form is protected within a firm gel matrix from which the protozoa will be released and infect the host after ingestion. Davis describes a preparation of viable sporulated coccidian oocysts for oral administration to poultry. Davis suggests modifying the consistency of the gel alginate by the inclusion of fillers (pg. 4, para. 2). Davis lists Ca2+ as possible filler for thickening the gel alginate.

U.S. Pat. No. 5,932,223 to Burke et al. discloses experimental findings that calcium does improve the stability of the rotaviral reassortments G1 and P1. More specifically, Burke teaches formulations prepared by dialysis of the rotavirus bulks into formulations without tissue culture. Based on his findings, Burke asserts that the use of Zn2+ in the presence or absence of Ca2+ dramatically increased the deactivation half-life of both the G1 and the P1 rotaviral reassortments. Significantly, Burke teaches that “the addition of divalent metals does not increase the thermal stability of rotavirus when formulated in a stabilizer containing tissue culture medium such as Williams E or Williams' modified E.” However, Burke does allow that “in preparing stabilized formulations of rotaviruses as described herein, it is preferable that sufficient levels of divalent metal ions be present.” Burke defines “sufficient levels of divalent metal ions” to mean “the final solution supplemented with 10 mM of either CaCl₂, MnCl₂, MgCl₂, ZnCl₂, or CaCl₂7+ZnCl₂ to yield a final concentration of 10 mM metal ion.” However, Burke does not teach or suggest a specific role of Ca2+ or excess Ca2+ in preparing a stabilized rotavirus vaccine formulation.

U.S. Pat. No. 6,616,931 B1 also to Burke further discloses liquid or lyophilized formulations of vaccines against rotavirus infections that include stabilizing components prior to manufacturing in order to increase potency and yield during the manufacturing process. The stabilization components claimed in Burke include a sugar, a phosphate, at least one carboxylate, and a non-ionic surfactant. Again, Burke teaches away from any role that excess levels of Ca2+ plays in maintaining the structural stability of the outer capsid of the rotavirus.

U.S. Pat. No. 6,165,773 to New et al. discloses methods of preparing viral particles for storage such that they retain infectivity, most specifically designed for polio virus. The general formulation claimed in New describes mixing virus particles with an amphiphile in an aqueous solution, removing the water (via freeze drying), and mixing the product with a hydrophobic solvent. However, New is silent on the use of Calcium to stabilize viral particles.

PCT International Pub. No. WO00/66710 A3 to Worral discloses a method for using two vacuum drying stages to preserve a virus or mycoplasm to provide a material that can be rehydrated to give a vaccine longer-lasting potency. The method involves desiccation without lyophilisation in a matrix of glassy trehalose having a residual moisture content of not greater than 2%. The method is meant to provide a faster process of producing preserved viral or mycoplasmic materials when compared to the method of freeze drying.

PCT Application International Publication Number No. WO 01/37804 A3 to Bronshtein discloses a method of preparing biological samples preserved as dry glassy powders and hydrophobic carriers for improving long term storage and delivery of viral and bacterial vaccines, vectors and cells at ambient or high temperatures.

U.S. Patent Application Pub. No. 2010/0120014 A1 also to Bronshtein, further discloses stabilization of biologics by immobilizing the biologics in dehydrated glass. Bronshtein's method teaches microspheres formulated using a cryo-encapsulating procedure which includes mixing drops of frozen preservation mixture (biologics mixed with solutions containing sodium alginate) with frozen drops of calcium solution and subsequent warming of the gel particles utilizing a vitrification process or a liquid to glass transition, Bronshtein specifically teaches away from the use of Ca2+ reporting, “We found that the presence of active Ca++ ion in the vaccine viral suspension strongly decreased the viral titer in both liquid state and in dry preserved state.” Bronshtein further teaches, “This phenomenon damaged the vaccine viruses which were encapsulated in alginate gel microspheres.” As a result of these findings, Bronshtein decreased the amounts of free Ca++ in the preservation solution to 0.05% from 0.25%. After making these adjustments, Bronshtein further concludes, “Although the survival rate after drying increases with decreasing CaCl₂ concentration in CS (calcium solution), we found that it is difficult to obtain good firm gel particles using CS with concentration of calcium chloride below 0.1%, We also are concerned that further decrease in Ca++ concentration could limit stability of the gel in the GI (gastrointestinal) tract and its protective role against gastric juice and bile.”

PCT Application Int. Pub. No. WO 2007/056847 A1 to Cigarini et al. discloses a stabilizing formulation for storing and preserving a virus, including a recombinant virus, for use as expression vectors, immunological formulations, and/or vaccines. The formulation is made up of a series of stabilizing compounds including a sugar, a preservative, a dispersing agent, a thermal stability agent, a buffer, and up to three distinct types of amino acids (arginine, alanine, serine, or glycine) without impacting the structural appearance of the lyophilized product. Cigarini discloses that the preparation may be suitable for rotavirus but teaches that a significant decrease in vitality of viral activity occurred when formulations were stored under stress conditions (para. 59). The stabilizing formulations of Cigarini are silent specifically on calcium and divalent metal ions in general.

U.S. Pat. No. 7,790,180 B2 to Colau et al. discloses a specific vaccine formulation for rotavirus and a method separating rotavirus variants to improve the potency of the live attenuated rotavirus. Colau also describes a formulation for a quick-dissolving table for immediate dissolution when placed on the tongue. One aspect of the formulation includes an antacid for neutralization including aluminum hydroxide, magnesium hydroxide and calcium carbonate. Colau teaches that CACO₃ associates well with rotavirus (col. 7, para. 4) to maintain rotavirus activity. Colau further teaches that this association can be aided by adding a viscous agent to the formulation. In one example, Colau teaches the use of CaCO₃ and xanthum gum. In a more specific example, Colau teaches the use of a formulation 60 mg of CaCO₃ per 1.5 ml of water or approx. 4 mM.

U.S. Patent Application Pub. No. US2011/0150940 A1 to Remon et al. discloses a dry powder composition for poultry vaccination delivered via inhalation. The dry powder composition taught by Remon includes a sugar and a biocompatible polymer. Remon further teaches a method for performing vaccination in poultry against an infection from a virus selected from a group which includes rotavirus. The dry powder poultry vaccine formulations of Remon are silent on Ca2+.

U.S. Patent Application Pub. No. US2008/0166372 A1 to Vande Velde discloses a formulation for a refrigerated live attenuated rotavirus vaccine for oral administration to a human infant containing a sugar, a carboxylate and reduced phosphates. Vande removes or decreases the phosphate component of the formula to the dosage size required to administer to infants. Vande Velde teaches a formulation including an adipate rotavirus liquid in the presence of additional calcium ion in two alternative forms: CaCl₂ and Ca(OH)₂, Vande Velde further teaches that “it may be beneficial to add calcium ions to the adipate rotavirus liquid formulation of the invention, as they may contribute to the stabilization of the rotavirus within the formulation.” However, Vande Velde fails to teach or suggest using calcium in a range greater than 1.9 mM or the use of high levels of calcium independent of the amount of phosphates. Further, Vande Velde fails to teach or suggest using higher levels of Ca2+ without the use of adipate acid as a pH control agent.

U.S. Patent Application Pub. No. US 2010/0226939 A1 to Truong-Le et al. discloses a formulation for stabilization of live viral vaccines to retain viability of rotavirus within liquid, dried, or lyophilized vaccines. Trong-Le specifically teaches employing Zn2+ in a concentration range from 0.5 mM to 20 mM, a carboxylate, a phosphate buffer, a sugar, and at least one strain of rotavirus in specific ranges for titer. Truong-Le further teaches that “particularly for storage of liquid formulations at high temperatures (above room temperature), the combinations of Zn2+ and Ca2+ can enhance stability. However, at more typical storage temperatures of about 25° C. (essentially “room temperature”), formulations with Zn2+ alone appear to be more stable than formulations with no divalent cations or with only Ca2+.”

For worldwide distribution of rotavirus vaccines, it is necessary to formulate vaccines that are stable under a variety of environmental conditions. Selected components used to stabilize vaccines are known. However, present formulations useful to stabilize rotavirus vaccines are limited and have not produced the robust efficacy needed to create a fully stabilized rotavirus vaccine.

### SUMMARY OF INVENTION

The present invention provides methods and compositions for stabilization of viruses. In particular, the compositions employ excess Ca2+ in combination with various other formulation constituents to stabilize rotavirus in live oral vaccine formulations. The present invention is based on the unique and surprising findings that the two structural proteins VP7 and VP4 which can be found on the outer capsid of rotavirus are bound by calcium cations. Therefore, the present invention presents a novel formulation for stabilizing the structural proteins that make up the rotavirus outer capsid through excessive amounts of Ca2+ in combination with higher solution viscosity that will ensure vaccine stability at high temperatures.

Definitions

Unless otherwise defined herein or below in the remainder of the specification, all technical and scientific terms used herein have meaning commonly understood by those of ordinary skill in the art to which the present invention belongs.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a,” “an” and “the” include the plural referents unless the content clearly dictates otherwise. Thus, for example, references to “a component” can include a combination of two or more components; e.g. a reference to “sugar” can include mixtures of sugars, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “about,” as used herein, indicates the value of a given quantity can include quantities ranging within 10% of the stated value, optionally, within 5% of the value, or in some embodiments within 1% of the value.

A “diluents,” as used herein, refers to a liquid or solution into which recited formulation constituents are solutes in the liquid formulations of the invention.

The term “cultivating,” as used herein refers to culturing a virus in an appropriate host cell.

The term “neutralizing,” as used herein with regard to stomach acid, refers to raising the stomach digestive juices to pH4 or above. Preferably, neutralizing in this context refers to raising the stomach digestive juices to a pH of about 6 or above.

The accompanying drawings, which are incorporated in and constitute part of the specification, illustrate various embodiments of the invention and together with the description, serve to explain the principles of the invention.

### BRIEF DESCRIPTION OF THE DRAWINGS

FIG. 1 shows a structure of rotavirus outer capsid.

FIG. 2 shows VP7 trimers are bound together by calcium.

FIG. 3 shows the stabilizing effect of high Ca2+ concentrations and viscous solutions.

FIG. 4 shows two stability curves of formulations according to the present invention.

### DETAILED DESCRIPTION OF THE INVENTION

Aspects of the invention are not limited in its application to the details of construction and the arrangement of components set forth in the preceding description or illustrated in the examples or in the drawings. Aspects of the invention are capable of other embodiments and of being practiced or carried out in various ways. In addition, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents.

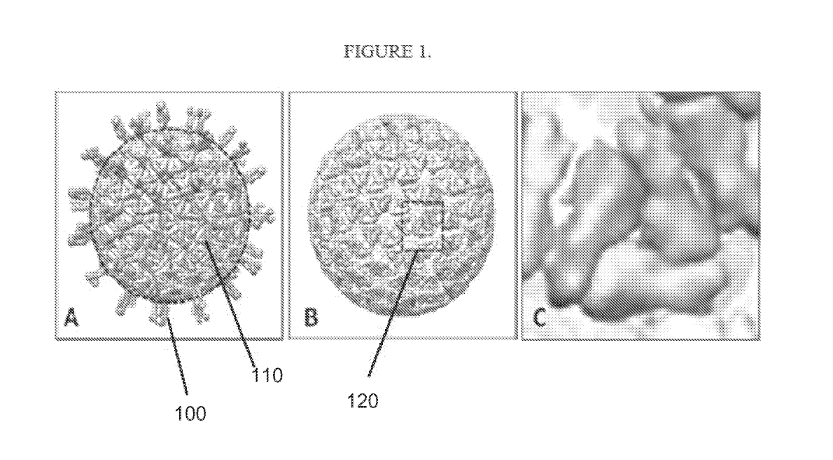

With reference now to FIG. 1, a structure of rotavirus outer capsid will now be discussed. As shown in FIG. 1, view A, the structure of the rotavirus outer capsid is based on two structural proteins on the surface of the virion: VP4 proteins 100 which appear as spikes and the VP7 proteins 110 which appear as triangles. VP4 binds to molecules on the surface of cells called receptors and drives the entry of the virus into the cell. VP4 has to be modified by a protease enzyme (found in the gut) into VP5* and VP8* before the virus is infectious. It determines how virulent the virus is and it determines the P-type of the virus. VP7 is a glycoprotein that forms the outer surface of the virion. Apart from its structural functions, it determines the G-type of the strain and, along with VP4, is involved in immunity to infection. As also shown in FIG. 1, view B, a perspective of a rotavirus capsid showing VP7 proteins only 130. As further shown in FIG. 1, view C, a close up view of three VP7 organized as a trimer.

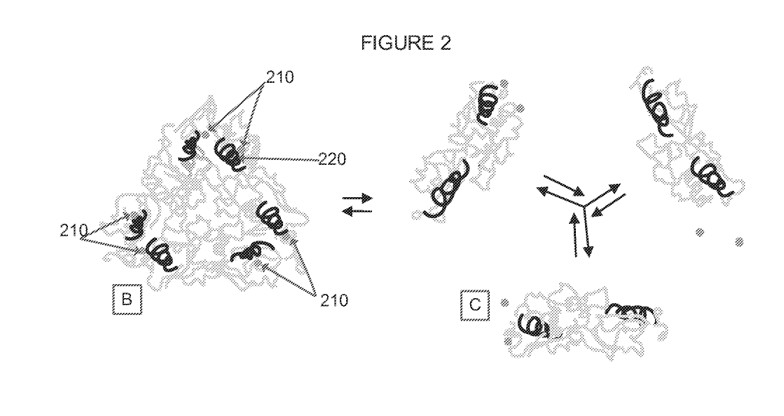

With reference now to FIG. 2, a view the VP7 trimer bound together by calcium, will now be discussed. As shown in FIG. 2, view B, VP7 protein trimer displayed in ribbon format showing six bound Ca2+ ions (210), and proximal Ca2+ binding pocket (220). As further shown in FIG. 2, view C, an exploded view of VP7 trimer shows that Ca2+ ions bind at protein-protein interface and that proximal binding pocket is defined by regions on separate proteins.

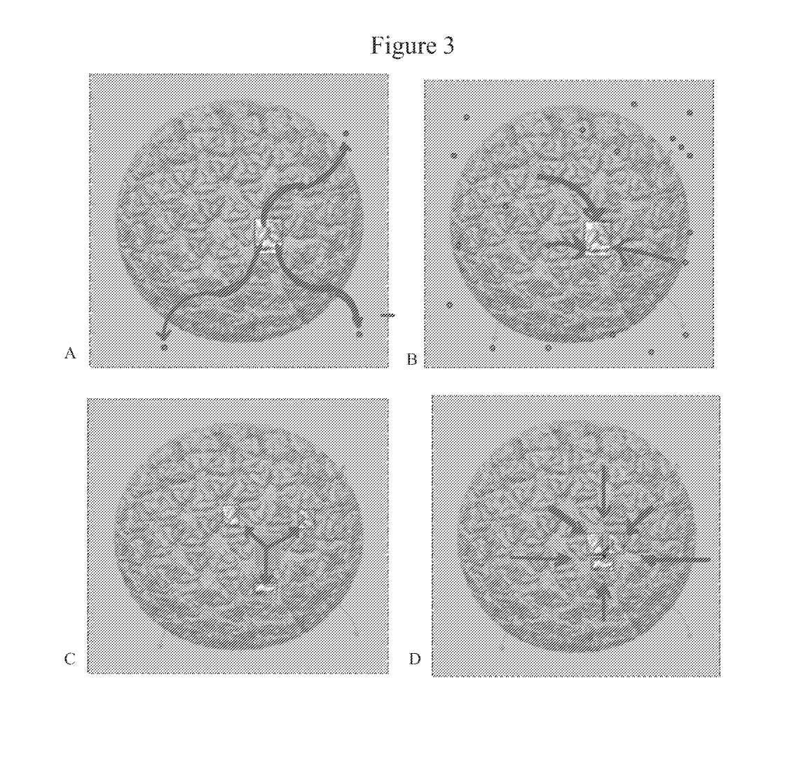

With reference now to FIG. 3, the stabilizing effect of high Ca2+ concentrations and viscous solutions will now be discussed. As shown in FIG. 3, view A, during high stress conditions (e.g. room temperature, liquid state, drying etc.) Ca2+ diffuses away which leaves the VP7 destabilized. As further shown in FIG. 3, view B, high Ca2+ concentrations lead to rapid replacement of the Ca2+ in the VP7 binding pocket thereby restabilizing the viral capsid structure. As further shown in FIG. 3, view C, in lower calcium environments, VP7 has lost Ca2+ and so the monomers diffuse away leading to loss of infective potency. As finally shown in FIG. 3, view D, high viscosity solutions oppose the outward diffusion of destabilized VP7 monomers thereby slowing the destabilization kinetics (e.g. buying time for Ca2+ to bind).

The premise of the present invention is that extremely stable preparations have high calcium and high viscosities. In one aspect of the present invention, the liquid vaccine formulation includes: at least one strain of rotavirus at a titer ranging from about 10³ IU/ml to about 10¹² IU/ml; Ca2+ concentration of at least 2 mM and up to 1M; a viscosity increasing agent (thickener at concentration such that the dynamic (absolute) viscosity of the solution is greater than about 1.5×10⁰ centapoise and up to about 1.5×10¹⁰ centapoise at 20° C.; Zn2+ in a concentration such that the ratio to Ca2+ concentration ([Zn2+]/[Ca2+]) is between 1.0×10⁻¹ and 1.0×10⁻¹⁰; at least one acid neutralizing agent ranging in concentration from about 0.1 mM to about 2 M; at least one diluent selected from the group consisting of: a tissue culture medium, saline and water; and about 0.001% to about 2% of a non-ionic surfactant. In one preferred embodiment of the present inventive formulations the pH of the formulation is within a range from about pH 5.0 to about pH 8.0.

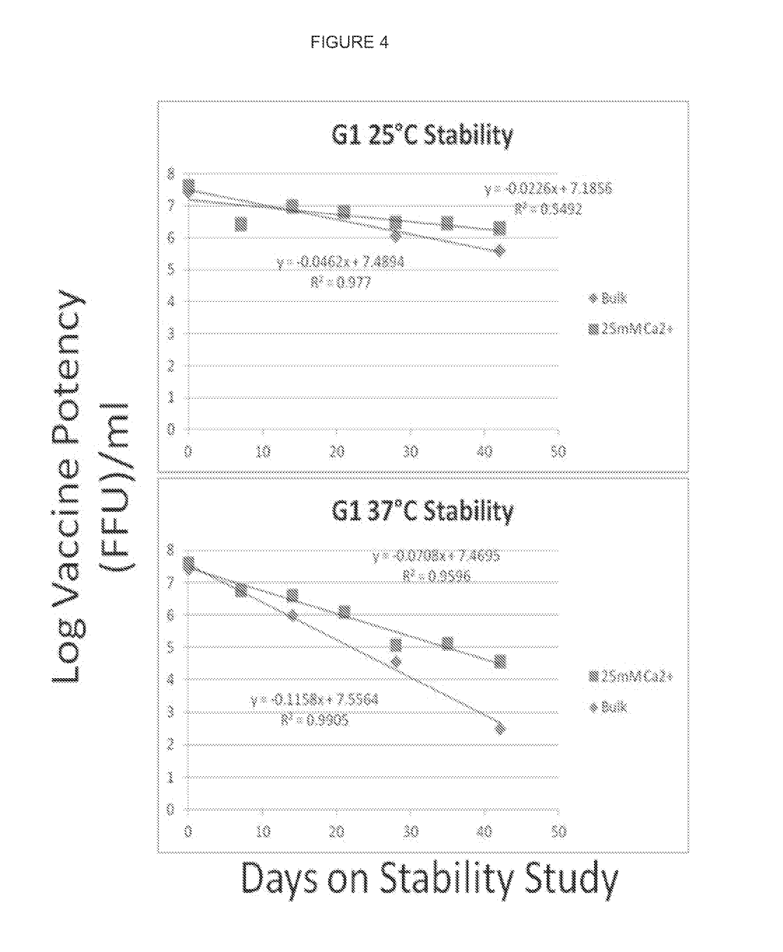

With reference now to FIG. 4, two stability curves of formulations according to the present invention will now be discussed. As shown in FIG. 4, two linear regression plots showing that high concentrations of calcium (2+) ions greatly improve the stability of rotavirus G1 serotype in liquid formulations. The two formulations consist of tissue culture media with two different calcium concentrations −1.5 mM (labeled “Bulk”) and 25 mM. The top figure is stability curves when formulations are held at 25° C. and the bottom figure is stability curves when formulations are held at 37° C. Fitting the data with linear regression trend lines (equations of the form y=mx+b) gives the rate of stability loss in the form of the slope parameter m. From the figure, it can be seen that formulations with 25 mM Ca2+ have nearly twice the stability as formulations with only Ca2+ from media over the course of six weeks.

Viscosity Agents

The viscosity agent is selected from the group consisting of: alginic acid, alginate, cellulose, carboxymethylcellulose, cyclodextrin, ethyl cellulose, galactose, gelatin, glucose, fructose, fucose, furanose, hemicellulose, hydroxy propyl cellulose, hydroxyl propyl methyl cellulose, hypromellose, lactose, maltose, mannitol, mannose, methyl celluloseinositol, N-acetylneuraminic acid-lactose, ribose, saccharose, sialic acid, sorbitol, starch, sucrose, trehalose, xylose. In one preferred embodiment, the viscosity agent may include a formulation parameter wherein the viscosity increasing agent is sucrose or alignate or hydroxyl methyl cellulose or gelatin; the viscosity is between about 1.5×10¹ centapoise and up to about 1.5×10⁷ centapoise at 20° C.

Neutralizing Agents

The formulation for the stabilization of rotavirus may further include a neutralizing component with a range of multiple parameters measured by the desired pH levels of the gastric juices in an individual's stomach either directly before or during administration. In a preferred embodiment of the present invention the formulation will include at least one acid neutralizing agent ranging in concentration from about 0.1 mM to about 2 M. The neutralizing agent may be selected from the group consisting of: acetate, adipate, bicarbonate, carbonate, citrate, glyceralphosphate, gluconate, formate, fumarate, lactate, malate, phosphate, succinate, tartarate. In a preferred embodiment, the acid neutralizing agents are acetate, adipate or lactate at a concentration between about 0.05 to about 0.7 M. In most preferred embodiments, the pH of the formulation ranges from about pH 5.0 to about pH 8.0.

Divalent Cations

Most preferred embodiments of the present inventive formulations include the presence of Ca2+ ions and Zn2+ ions in specified ranges based on the ratio of divalent cations. In one preferred embodiment, Zn2+ is provided in a concentration such that the ratio to Ca2+ concentration ([Zn2+]/[Ca2+]) is between 1.0×10-1 and 1.0×10-10. However, Zn2+/Ca2+ may go from 10̂-1 to 0. Preferably, according to some embodiments of the present invention Ca2+ may be at least 10× the Zn2+ but may also be 100× or 1,000,00×. Also according to certain formulations of the present invention, Zn2+ may not be present at all which makes the ratio 0.

Diluent

In preferred embodiments, the formulations may include at least one of the following diluents: a tissue culture medium, saline and water.

Non-Ionic Surfactant

Formulations of the invention may benefit from the presence of a non-ionic surfactant in the formulation. In preferred embodiments, the non-ionic surfactant is ingestible. In some preferred embodiments, the formulations range from about 0.001% to about 2% of a non-ionic surfactant. Preferably, the non-ionic surfactant is selected from the group consisting of: a polysorbate, a polyoxyethylene alkyl ether, a nonaethylene glycol octylphenyl ether, a hepatethytene glycol octylphenyl ether, a sorbitan trioleate, and a polyoxyethylene-polyoxypropyiene block copolymer. In some preferred embodiments, the non-ionic surfactant concentration may range from about 0.005% to about 0.1%. Preferred embodiments, particularly pertaining to the liquid formulations and liquid-gel formulations, may include a gelatin in the range of 0.5% to about 5% or from about 0.001% to about 2% of anon-ionic surfactant.

Methods of Desiccation

In some embodiments, the rotavirus formulation of the present invention may be prepared in a solid or semi-solid form. For example, the liquid-get formulation can be cast into thin films which are easily packaged, shipped and administered. Another means of preserving the liquid formulation or liquid-gel formulation of the present invention is through cryodesiccation or lyophilizing. As outlined in the background of the invention above, lyophilizing is known to be costly, time consuming and to degrade the potency of the sample. Another preferred means of dehydrating the liquid or liquid-gel formulations is through the means of spray-drying which will produce a dry powder from the liquid or liquid-gel formulation by rapidly drying with a hot gas. The dry formulations can be compressed into a pill form. In some embodiments, the liquid or liquid-gel formulations can be dried through the process of fluid-bed drying which involves drying, cooling, agglomeration, granulation, and coating of particulate materials. Additionally, in some embodiments it may be preferred that the liquid or liquid-gel formulations of the present invention undergo the process of air-drying at temperatures above 0° C.

Administering an Oral Rotavirus Vaccine

Administering the formulations of the present invention can include oral administration of the stabilized vaccine to an individual. A method of administering an oral rotavirus vaccine to an individual may preferably comprise neutralizing the individuals stomach acid by orally administering an acid neutralizing agent to the individual. This can be accomplished by administration of an antacid, such as calcium carbonate or magnesium carbonate, before or during administration of the vaccine. Optionally, the vaccine itself can be formulated to include sufficient pH buffer capacity to raise the individual's stomach interior above pH 4. In preferred embodiments, the individual's stomach pH may be raised to about pH6 or pH7. In one preferred embodiment, the formulation for orally administering the rotavirus vaccine comprises at least 4 mM Ca2+ and a viscosity of at least 1.5 centapoise.

### EXAMPLES

### Example 1

A liquid vaccine formulation including: at least one strain of rotavirus at a titer ranging from about 10³ IU/ml to about 10¹² IU/ml; Ca2+ concentration greater than 4 mM; a viscosity increasing agent such that the viscosity is at least about 1.5×10¹ centapoise and up to about 1.5×10⁴ centapoise at 20° C.; and an acid neutralizing compound at a concentration between about 0.1 M to about 0.7 M; wherein the formulation pH is about pH 6.0 to about pH 7.5.

### Example 2

A method of stabilizing a rotavirus in a liquid formulation, the method includes the steps of: titering at least one strain of rotavirus at a titer ranging from about 10³ IU/ml to about 10¹² IU/ml; bathing the titered strain of rotavirus Ca2+ in a concentration greater than 4 mM; adding a viscosity increasing agent such that the viscosity is at least about 1.5×10¹ centapoise and up to about 1.5×10¹⁰ centapoise at 20° C.; and neutralizing the mixture with a neutralizing compound at a concentration between about 0.1 mM to about 1 M wherein the formulation pH is about pH 5.0 to about pH 8.0.

### Example 3

A liquid-gel vaccine formulation includes: at least one strain of rotavirus at a a titer ranging from about 10³ IU/ml to about 10¹² IU/ml; a Ca2+ a concentration greater than 4 mM; a viscosity increasing agent such that the viscosity is at least about 1.5×10⁴ centapoise and up to about 1.5×10⁷ centapoise at 20° C.; an acid neutralizing compound at a concentration between about 0.1 to about 0.7 M wherein the formulation pH is about pH 6.0 to about pH 7.5.

### Example 4

The liquid vaccine formulation includes: at least one strain of rotavirus at a titer ranging from about 10³ (IU)/ml to about 10¹² IU/ml and a Ca2+ concentration of at least 2 mM and up to 1 M; a viscosity increasing agent (thickener) at concentration such that the dynamic (absolute) viscosity of the solution is greater than about 1.5×10⁰ centapoise and up to about 1.5×10¹⁰ centapoise at 20° C.; Zn2+ in a concentration such that the ratio to Ca2+ concentration ([Zn2+]/[Ca2+]) is between 1.0×10⁻¹ and 1.0×10⁻¹⁰; at least one acid neutralizing agent ranging in concentration from about 0.1 mM to about 2 M, at least one diluent selected from the group consisting of: a tissue culture medium, saline and water; and about 0.001% to about 2% of a non-ionic surfactant. in one preferred embodiment, the pH of the formulation is preferably adjusted to orange from about pH 5.0 to about pH 8.0.

While the above descriptions regarding the present invention contains much specificity, these should not be construed as limitations on the scope, but rather as examples. Many other variations are possible. Accordingly, the scope should be determined not by the embodiments illustrated, but by the appended claims and their legal equivalents.

1. A liquid formulation comprising:
at least one strain of rotavirus at a titer ranging from about 10³ IU/ml to about 10¹² IU/ml;
a Ca2+ concentration of at least 2 mM to about 1M and a viscosity increasing agent (thickener) at a concentration such that the dynamic (absolute) viscosity of the solution is greater than about 1.5×10⁰ centapoise and up to about 1.5×10¹⁰ centapoise at 20° C.;
a Zn2+ in a concentration such that the ratio to Ca2+ concentration ([Zn2+]/[Ca2+]) is between 1.0×10⁻¹ and 1.0×10⁻¹⁰ wherein the Zn2+ in a concentration such that the ratio to Ca2+ concentration ([Zn2+]/[Ca2+]) ranges from 0:1 to 1:1,000,000; and
at least one acid neutralizing agent ranging in concentration from about 0.1 mM to about 2 M.

2. (canceled)

3. The formulation of claim 1, further comprising at least one diluent selected from the group consisting of: a tissue culture medium, saline or water.

4. The formulation of claim 1, further comprising from about 0.001% to about 2% of a non-ionic surfactant.

5. The formulation of claim 1, wherein pH of the formulation ranges from about pH 5.0 to about pH 8.0.

6. The formulation according to claim 1, wherein the viscosity increasing agent is selected from the group consisting of: alginic acid, alginate, cellulose, carboxymethylcellulose, cyclodextrin, ethyl cellulose, galactose, gelatin, glucose, fructose, fucose, furanose, hemicellulose, hydroxy propyl cellulose, hydroxyl propyl methyl cellulose, hypromellose, lactose, maltose, mannitol, mannose, methyl celluloseinositol, N-acetylneuraminic acid-lactose, ribose, saccharose, sialic acid, sorbitol, starch, sucrose, trehalose, or xylose.

7. The formulation according to claim 1, wherein the acid neutralizing agent is selected from the group consisting of: acetate, adipate, bicarbonate, carbonate, citrate, glyceralphosphate, gluconate, formate, fumarate, lactate, malate, phosphate, succinate, or tartarate.

8. The formulation according to claim 1, wherein the viscosity is between about 1.5×10¹ centapoise and up to about 1.5×10⁷ centapoise at 20° C.

9. The formulation of claim 1, wherein the viscosity increasing agent is selected from the group consisting of: sucrose, alignate, hydroxyl methyl cellulose or gelatin;
the viscosity is between about 1.5×10¹ centapoise and up to about 1.5×10⁷ centapoise at 20° C.; and
the acid neutralizing agents is selected form the group consisting of: acetate, adipate or lactate at a concentration between about 0.05 to about 0.7M.

10. The formulation according to claim 1, wherein the non-ionic surfactant is selected from the group consisting of: a polysorbate, polyoxyethylene alkyl ether, nonaethylene glycol octylphenyl ether, a hepatethylene glycol octylphenyl ether, a sorbitan trioleate, or a polyoxyethylene-polyoxypropylene block copolymer.

11. The formulation according to claim 3, wherein the surfactant concentration ranges from about 0.005% to about 0.1%.

12. A liquid vaccine formulation comprising:
at least one strain of rotavirus at a titer ranging from about 10³ IU/ml to about 10¹² IU/ml;
a Ca2+ concentration greater than 4 mM;
a viscosity increasing agent such that the viscosity is at least about 1.5×10¹ centapoise and up to about 1.5×10⁴ centapoise at 20° C.; and
an acid neutralizing compound at a concentration between about 0.1 to about 0.7 M wherein the formulation pH is about pH 6.0 to about pH 7.5.

13. The vaccine formulation of claim 12, wherein the formulation further comprises Zn2+ at a concentration such that the ratio to Ca2+ concentration ([Zn2+]/[Ca2+]) is between 1.0×10⁻¹ and 1.0×10⁻¹⁰.

14. The vaccine formulation of claim 12, wherein the formulations further comprise from about 0.5% to about 5% of gelatin or from about 0.001% to about 2% of a non-ionic surfactant.

15. The vaccine formulation of claim 12, wherein the viscosity increasing agent is selected from the group consisting of: alginic acid, alginate, cellulose, carboxymethylcellulose, cyclodextrin, ethyl cellulose, galactose, gelatin, glucose, fructose, fucose, furanose, hemicellulose, hydroxy propyl cellulose, hydroxyl propyl methyl cellulose, hypromellose, lactose, maltose, mannitol, mannose, methyl celluloseinositol, N-acetylneuraminic acid-lactose, ribose, saccharose, sialic acid, sorbitol, starch, sucrose, trehalose, or xylose.

16. A liquid-gel vaccine formulation comprising:
at least one strain of rotavirus at a titer ranging from about 10³ IU/ml to about 10¹² IU/ml;
a Ca2+ a concentration greater than 4 mM;
a viscosity increasing agent such that the viscosity is at least about 1.5×10⁴ centapoise and up to about 1.5×10⁷ centapoise at 20° C.;
a Zn2+ in a concentration such that the ratio to Ca2+ concentration ([Zn2+]/[Ca2+]) is between 1.0×10⁻¹ and 1.0×10⁻¹⁰;
a non-ionic surfactant wherein the non-ionic surfactant agent comprises from about 0.5% to about 5% of gelatin or from about 0.001% to about 2% of a non-ionic surfactant; and
an acid neutralizing compound at a concentration between about 0.1 to about 0.7 M wherein the formulation pH is about pH 6.0 to about pH 7.5.

17. (canceled)

18. (canceled)

19. The vaccine formulation of claim 16, wherein the viscosity increasing agent is selected from the group consisting of: alginic acid, alginate, cellulose, carboxymethylcellulose, cyclodextrin, ethyl cellulose, galactose, gelatin, glucose, fructose, fucose, furanose, hemicellulose, hydroxy propyl cellulose, hydroxyl propyl methyl cellulose, hypromellose, lactose, maltose, mannitol, mannose, methyl celluloseinositol, N-acetylneuraminic acid-lactose, ribose, saccharose, sialic acid, sorbitol, starch, sucrose, trehalose, or xylose.

20. (canceled)
